(19) United States
(12) Patent Application Publication
(10) Pub. No.: US 2005/0282146 A1
(43) Pub. Date: Dec. 22, 2005

(54) Methods to predict additional nodal metastases in breast cancer patients

(76) Inventors: Michael Kattan, Cleveland Heights, OH (US); Kimberly J. Van Zee, New York, NY (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/996,172

(22) Filed: Nov. 23, 2004

Related U.S. Application Data
(60) Provisional application No. 60/525,325, filed on Nov. 26, 2003.

(51) Int. Cl. C12Q 1/00; G06F 19/00; G01N 33/48; G01N 33/50

(52) U.S. Cl. 435/4; 702/20

(57) ABSTRACT

A method and system to predict the likelihood of additional nodal metastases in breast cancer patients with a positive sentinel node biopsy.

### CROSS-REFERENCE TO RELATED APPLICATIONS

This application claims the benefit of the filing date of U.S. application Ser. No. 60/525,325, filed Nov. 26, 2003, under 35 U.S.C. § 119(e), the disclosure of which is incorporated by reference herein.

### BACKGROUND

The sentinel lymph node (SLN) biopsy procedure has been validated by numerous studies and found to be accurate for assessing regional lymph node involvement (Giuliano et al., 1997; Albertini et al., 1996; Veronesi et al., 1997; O'Hea et al., 1998; Krag et al., 1998; Veronesi et al., 1999). For those with a negative SLN biopsy by histopathologic exam, the risk of “missed” axillary disease is extremely low (Giuliano et al., 2000; Turner et al., 1997). Therefore, SLN biopsy alone, without complete axillary lymph node dissection (ALND), has been adopted at many institutions as an accurate method of staging the axilla while avoiding much of the morbidity associated with a complete ALND. However, the standard of care for breast cancer patients with sentinel lymph node (SLN) metastases remains complete axillary lymph node dissection (ALND). Yet many question the need for completion ALND in every patient with detectable SLN metastases, particularly those in whom the perceived risk of additional disease is low (Chu et al., 1999; Kamath et al., 2001).

Proponents of completion ALND after a positive SLN biopsy argue that the further axillary clearance is critical to further management. The total number of involved nodes is important prognostic information, as an increasing number of positive nodes portends a worse survival (Cabanes et al., 1992; Moore et al., 1997; Carter et al., 1989). This is reflected in the new American Joint Committee on Cancer (AJCC) 6th edition staging system (2002), where the number of positive nodes defines N1, N2, and N3 disease and ultimately the stage to which the patient is assigned. In addition, proponents of complete ALND after positive SLN biopsy argue that the additional information can benefit patients by guiding decisions about adjuvant chemotherapy. For the approximately one-half of patients in whom there is residual nodal disease, it is also argued that complete ALND can influence survival via local-regional control of the axilla (Sosa et al., 1998; Hayward et al., 1987; Osteen et al., 1985), thereby eliminating a potential site of recurrent disease and, ultimately, a source for distant disease. A meta-analysis of randomized trials found a 5.4% survival benefit associated with ALND for clinically node-negative patients (Orr, 1999).

Opponents of complete ALND after positive SLN biopsy argue that the therapeutic benefit of complete ALND is minimal (Cady, 1997). Furthermore, approximately 50% of patients with positive SLNs are found to have no other nodal metastases (Giuliano et al., 1997; Albertini et al., 1996; Veronesi et al., 1997; O'Hea et al., 1998; Krag et al., 1998; Veronesi et al., 1999; Guiliano et al., 1994; Reynolds et al., 1999; Teng et al., 2000; Abdessalam et al., 2001; Rahusen et al., 2001). Therefore, many patients are possibly undergoing “unnecessary” ALND, with no additional therapeutic benefit or further staging information. It is also argued that because patients with SLN metastases will generally receive systemic therapy regardless of the presence of any additional nodal metastases, any residual disease does not influence choice of therapy and may itself be eradicated by the systemic therapy. In addition, radiation therapy after breast-conserving surgery may contribute to control of any additional nodal disease. It is this debate that physicians and their patients are faced with in the office setting when a positive SLN is discovered on final pathology.

Several groups have identified histopathologic variables of the primary tumor and its metastasis that can influence risk of having additional disease in the non-SLNs (Chu et al., 1999; Kamath et al., 2001; Reynolds et al., 1999; Teng et al., 2000; Abdessalam et al., 2001; Rahusen et al., 2001) (FIG. 1). Size of the primary tumor and size of the SLN metastasis are the two variables most commonly analyzed. Weiser et al. (2001) reported that size of the primary tumor and of the SLN metastasis are significantly predictive of likelihood of additional, non-SLN metastases. Most other studies that examined at least one of these two variables found a statistically significant correlation with risk of additional, non-SLN disease (Chu et al., 1999; Kamath et al., 2001; Reynolds et al., 1999; Teng et al., 2000; Abdessalam et al., 2001; Rahusen et al., 2001; Turner et al., 2000; Wong et al., 2001; Viale et al., 2001; Sachdev et al., 2002). Yet most have not been able to identify a subset that has no risk of additional disease in the non-SLNs (Kamath et al., 2001; Teng et al., 2000; Abdessalam et al., 2001; Rahusen et al., 2001; Wong et al., 2001; Viale et al., 2001) and in studies that have identified favorable subsets with an apparent negligible risk of additional nodal disease have all involved very small subsets (i.e., 5-24 patients) (Chu et al., 1999; Reynolds et al., 1999; Weiser et al., 2001; Jakub et al., 2002; Czerniecki et al., 1999).

Furthermore, it is difficult to estimate the risk of additional, non-SLN metastases for an individual patient using the published literature. First, the estimates of risk for any given characteristic vary considerably among studies. FIGS. 2-5 show reported incidences by primary tumor size, size of SLN metastasis, presence of lymphovascular invasion (LVI) in the primary tumor, and in immunohistochemically (IHC) detected SLN metastases, respectively. This variation in reported incidences may be attributable to relatively small sample sizes or the result of differences among the study populations in terms of other variables influencing risk. Secondly, it is difficult to apply risk estimates to several patient characteristics simultaneously because of the generally univariate method of reporting found in the literature.

What is needed is an improved method to predict additional nodal metastases in breast cancer patients with a positive sentinel node biopsy.

### SUMMARY OF THE INVENTION

The invention provides methods, apparatus and nomograms to predict or determine the probability (likelihood) of additional nodal metastases in a breast cancer patient with a positive sentinel node biopsy. In one embodiment, the invention includes correlating the value or score from clinical and/or pathological data of the patient, for example, in a nomogram, to predict the likelihood of additional nodal metastasis. For instance, the methods, apparatus or nomograms may be employed after biopsy, e.g., after sentinel lymph node (SNL) biopsy, and before adjuvant therapy, and optionally prior to surgery for breast cancer, such as prior to completion axillary lymph node dissection, to predict the risk of additional nodal metastases in the patient.

As described herein, pathologic features of the primary tumor and sentinel lymph node metastases of 702 patients who underwent completion ALND were assessed with multivariable logistic regression to predict the presence of additional disease in the non-sentinel lymph nodes of these patients. A nomogram was created using pathologic size, tumor type and nuclear grade, lymphovascular invasion, multifocality, and estrogen receptor status of the primary tumor, as well as the method of detection of sentinel lymph node metastases, the number of positive sentinel lymph nodes, and the number of negative sentinel lymph nodes. The model was subsequently applied prospectively to 373 patients. The nomogram for the retrospective population was accurate and discriminating, with an area under the receiver operations curve (ROC) of 0.76. When applied to the prospective group, the model accurately predicted likelihood of non-sentinel lymph node disease (ROC of 0.77). Thus, a user friendly nomogram is provided which employs information commonly available to surgeons to easily and accurately calculate the likelihood of additional, non-sentinel lymph node metastases in an individual patient.

As also described herein, pathologic features of the primary tumor and SLN metastases of 33 patients who underwent completion ALND were assembled and presented to 17 breast cancer specialists. Their predictions for each woman were recorded and compared with results from the nomogram. The area under the ROC curve was computed for the nomogram, each clinician, and the clinicians as a whole. Subsequently, clinicians were presented with clinical information of 8 patients and asked if they would perform a completion ALND before and after being presented with the nomogram prediction. The predictive model achieved an area under the ROC curve of 0.72 when applied to the test data set of 33 patients. In comparison, the clinicians as a group were associated with an area under the ROC curve of 0.54 (P<0.01 vs. nomogram). When examined individually, one of the 17 clinicians outperformed the predictive model. Thus, the predictive model appeared to substantially outperform clinical experts.

Therefore, various factors from the primary tumor and/or sentinel lymph nodes, e.g., from a biopsy, in a breast cancer patient are employed to predict the likelihood of additional nodal metastases in that patient. In one embodiment, the prognosis is based on a computer derived analysis of data of the amount, level or other value (score) for one or more, e.g., two, three, four or more, of those factors. Data may be input manually or obtained automatically from an apparatus for measuring the amount, level or value of one or more factors.

Accordingly, the invention provides a method, apparatus, e.g., a computerized tool, and nomogram to predict the likelihood of additional nodal metastases in breast cancer patients with a positive sentinel node biopsy, which is useful for counseling breast cancer patients. In one embodiment, the invention provides a method to determine a likelihood of additional nodal metastases in a breast cancer patient with a positive sentinel node biopsy. The method includes detecting or determining one or more factors, e.g., at least two, three, four, five, six, seven, eight or more factors, of the patient including but not limited to pathological size of the breast carcinoma, number of positive sentinel lymph nodes, number of negative sentinel lymph nodes, method for histopathologic evaluation of the sentinel lymph node, tumor type and nuclear grade, estrogen receptor status, presence or absence of lymphovascular invasion, and/or presence or absence of multifocality in the primary tumor, as well as expression and/or genomic data of the patient. The factors of the patient are correlated to the likelihood of additional nodal metastases in that patient.

Further provided is a method to determine the likelihood of additional nodal metastases in a breast cancer patient with a positive sentinel node biopsy, which includes inputting test information to a data input means. The information includes one or more factors, e.g., at least two, three, four, five, six, seven, eight or more factors, of the patient factors including but not limited to pathological size of the breast carcinoma, number of positive sentinel lymph nodes, number of negative sentinel lymph nodes, method of histopathologic evaluation of the sentinel lymph node, tumor type and nuclear grade, estrogen receptor status, presence or absence of lymphovascular invasion, and/or presence or absence of multifocality. A software is executed for analysis of the test information, and the test information analyzed so as to provide the likelihood of additional nodal metastases in the patient.

Also provided is a method for predicting the likelihood of additional nodal metastases in a breast cancer patient with a positive sentinel node biopsy. The method includes correlating one or more factors for the patient to a functional representation of one or more factors determined for each of a plurality of persons previously diagnosed with invasive breast cancer, having a positive sentinel lymph node biopsy and subjected to completion axillary lymph node dissection, so as to yield a value for total points for the patient. The factors for each of the plurality of persons is correlated with the likelihood of additional nodal metastases for each person in the plurality, wherein the one or more factors include but are not limited to the pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or presence or absence of multifocality. The functional representation includes a scale for two or more of the factors, a total points scale, and a predictor scale. The scales for pathological size of the carcinoma, number of positive sentinel lymph nodes, number of negative sentinel lymph nodes, method for histopathogic evaluation of the sentinel lymph nodes, tumor type and nuclear grade, estrogen receptor status, lymphovascular invasion, and/or multifocality, each have values on the scales which can be correlated with values on the points scale, and the total points scale has values which may be correlated with values on the predictor scale. The value on the total points scale for the patient is correlated with a value on the predictor scale to predict the likelihood of additional nodal metastases in the patient.

The invention also provides an apparatus. In one embodiment, the apparatus includes a data input means, for input of test information for one or more factors from a breast cancer patient with a positive sentinel node biopsy, which factors include but are not limited to pathological size of the carcinoma, number of positive sentinel lymph nodes, number of negative sentinel lymph nodes, method for histopathogic evaluation of the sentinel lymph nodes, tumor type and nuclear grade, estrogen receptor status, the presence or absence of lymphovascular invasion, and/or the presence or absence of multifocality in the primary tumor, a processor, executing a software for analysis of each factor. The software analyzes the factors for the patient and provides the likelihood of additional nodal metastases in the patient.

In one embodiment, the invention provides an apparatus for predicting the likelihood of additional nodal metastases in a breast cancer patient with a positive sentinel node biopsy. The apparatus includes a correlation of one or more factors for each of a plurality of persons previously diagnosed with invasive breast cancer, having a positive sentinel lymph node biopsy and having completion axillary lymph node dissection, with the likelihood of additional nodal metastases for each person of the plurality of persons. The one or more factors include but are not limited to pathological size of the invasive carcinoma, number of positive sentinel lymph nodes, number of negative sentinel lymph nodes, method for histopathogic evaluation of the sentinel lymph nodes, nuclear grade, estrogen receptor status, presence or absence of lymphovascular invasion, and/or presence or absence of multifocality of the primary tumor. The apparatus also includes a means for comparing an identical set of factors determined from a patient having breast cancer and a positive sentinel lymph node biopsy to the correlation to predict the likelihood of additional nodal metastases in the patient.

Further provided is an apparatus for predicting the likelihood of additional nodal metastases in a breast cancer patient with a positive sentinel node biopsy. The apparatus includes a scale for one or more factors of the patient factor such as pathological size of the breast carcinoma, number of positive sentinel lymph nodes, number of negative sentinel lymph nodes, method for histopathogic evaluation of the sentinel lymph nodes, nuclear grade, estrogen receptor status, presence or absence of lymphovascular invasion, and/or presence or absence of multifocality in the primary tumor, a points scale, a total points scale and a predictor scale. The scales for pathological size of the breast carcinoma, number of positive sentinel lymph nodes, number of negative sentinel lymph nodes, method for histopathogic evaluation of the sentinel lymph nodes, nuclear grade, estrogen receptor status, presence or absence of lymphovascular invasion, and/or presence or absence of multifocality, each has values on the scales, and the scales for pathological size of the breast carcinoma, number of positive sentinel lymph nodes, number of negative sentinel lymph nodes, method for histopathogic evaluation of the sentinel lymph nodes, nuclear grade, estrogen receptor status, lymphovascular invasion, and/or multifocality, are disposed so that each of the values can be correlated with values on the points scale. The total points scale has values scale and is disposed on the solid support with respect to the predictor scale so that the values on the total points scale may be correlated with values on the predictor scale, such that the values on the points scale correlating with pathological size of the breast carcinoma, number of positive sentinel lymph nodes, number of negative sentinel lymph nodes, method for histopathogic evaluation of the sentinel lymph nodes, nuclear grade, estrogen receptor status, presence or absence of lymphovascular invasion, and/or presence or absence of multifocality of the patient can be added together to yield a total points value, and the total points value can be correlated with the predictor scale to predict the likelihood of additional nodal metastases in the patient.

Also provided is a system which includes a processor, an input device, an output device, a storage device, a database wherein the database includes data collected from a plurality of patients previously diagnosed with invasive breast cancer, having a positive sentinel lymph node biopsy and subjected to completion axillary lymph node dissection, and software operable on the processor to receive input from the input device. The input includes one or more factors for determining the likelihood of additional nodal metastases in a breast cancer patient with a positive sentinel node biopsy factors such as pathological size of the breast carcinoma, number of positive sentinel lymph nodes, number of negative sentinel lymph nodes, method for histopathologic evaluation of the sentinel lymph node, nuclear grade, estrogen receptor status, presence or absence of lymphovascular invasion, and/or presence or absence of multifocality in the primary tumor. The received input is correlated with the collected data from the plurality of patients to determine a likelihood of additional nodal metastases the patient.

### BRIEF DESCRIPTION OF THE DRAWINGS

FIG. 1. Studies reporting predictors of non-SLN metastases in patients with a positive SLN biopsy.

FIG. 2. Studies reporting incidence of non-SLN metastases in axillae with positive SLN by primary tumor size.

FIG. 3. Studies reporting incidence of non-SLN metastases in axillae with positive SLN by presence of LVI in primary tumor.

FIG. 4. Studies reporting incidence of non-SLN metastases in axillae with positive SLN by size of SLN metastases.

FIG. 5. Studies reporting incidence of non-SLN metastases in patients with immunohistochemically-detected SLN metastases.

FIG. 6A. Nomogram to predict likelihood of additional, non-SLN metastases in a patient with a positive SLN. NUCGRADE, tumor type and nuclear grade (ductal, nuclear grade I; ductal, nuclear grade II; ductal, nuclear grade III; lobular); LVI, lymphovascular invasion; MULTIFOCAL, multifocality of primary tumor; ER, estrogen-receptor status; NUMNEGSLN, number of negative SLNs; NUMSLNPOS, number of positive SLNs; PATHSIZE, pathologic size, defined in cm; and METHDETECT, method of detection of SLN metastases (Frozen, Routine, Serial HE, IHC). The first row (POINTS) is the point assignment for each variable. Rows 2-9 represent the variables included in the model. For an individual patient, each variable is assigned a point value (uppermost scale, POINTS) based on the histopathologic characteristics. A vertical line is made between the appropriate variable value and the POINTS line. The assigned points for all eight variables are summed and the total is found in row 10 (TOTAL POINTS). Once the total is located, a vertical line is made between TOTAL POINTS and the final row, row 11 (Predicted Probability of +Non-SLN).

FIG. 6B. Nomogram to predict likelihood of additional, non-SLN metastases in a patient with a positive SLN.

The first row (POINTS) is the point assignment for each variable. Rows 2-9 represent the variables included in the model. For an individual patient, each variable is assigned a point value (uppermost scale, POINTS) based on the histopathologic characteristics. A vertical line is made between the appropriate variable value and the POINTS line. The assigned points for all eight variables are summed and the total is found in row 10 (TOTAL POINTS). Once the total is located, a vertical line is made between TOTAL POINTS and the final row, Row 11 (Predicted Probability of +LN).

FIG. 7. Calibration plot for nomogram with frozen section information. The nomogram developed using the retrospective group of patients (n=702) was applied to the prospective group (n=373). A histogram of the calculated probabilities for the prospective population is shown along the horizontal axis. These 373 patients are grouped in deciles of their predicted probabilities, and the actual incidence of additional, non-SLN metastases was calculated for each decile. The vertical axis represents the actual, observed incidence (Actual Probability), and the horizontal axis represents the probability calculated by the nomogram (Predicted Probability). For each decile of the prospective group, a triangle is plotted to show actual probability. If the model were perfect, all triangles would lie on the dotted line with a slope of 1.

FIG. 8. Nomogram without frozen section information, for use when frozen section is not done at the time of SLN biopsy.

FIG. 9. Calibration plot for nomogram without frozen section data. The nomogram developed using the retrospective group of patients (n=702) was applied to the prospective group (n=373). A histogram of the calculated probabilities for the prospective population is shown along the horizontal axis. These 373 patients are grouped in deciles of their predicted probabilities, and the actual incidence of additional, non-SLN metastases was calculated for each decile. The vertical axis represents the actual, observed incidence (Actual Probability), and the horizontal axis represents the probability calculated by the nomogram (Predicted Probability). For each decile of the prospective group, a triangle is plotted to show actual probability. If the model were perfect, all triangles would lie on the dotted line with a slope of 1.

FIG. 10. Block diagram of a computer system useful to predict additional modal metastases in breast cancer patients with a positive sentinel node biopsy.

FIG. 11. Information processing system useful to predict additional nodal metastases in breast cancer patients with a positive sentinel node biopsy.

FIG. 12. Machine readable medium useful to predict additional nodal metastases in breast cancer patients with a positive sentinel node biopsy.

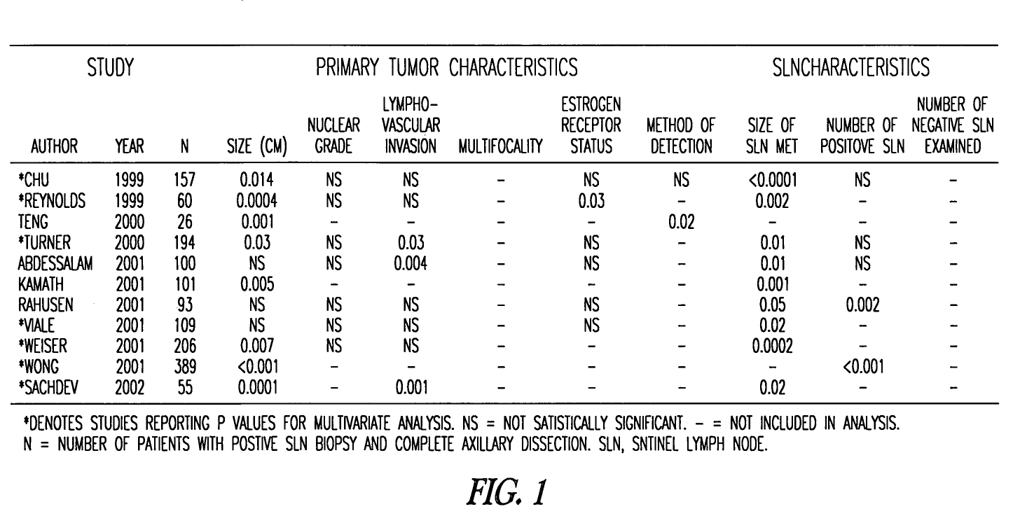

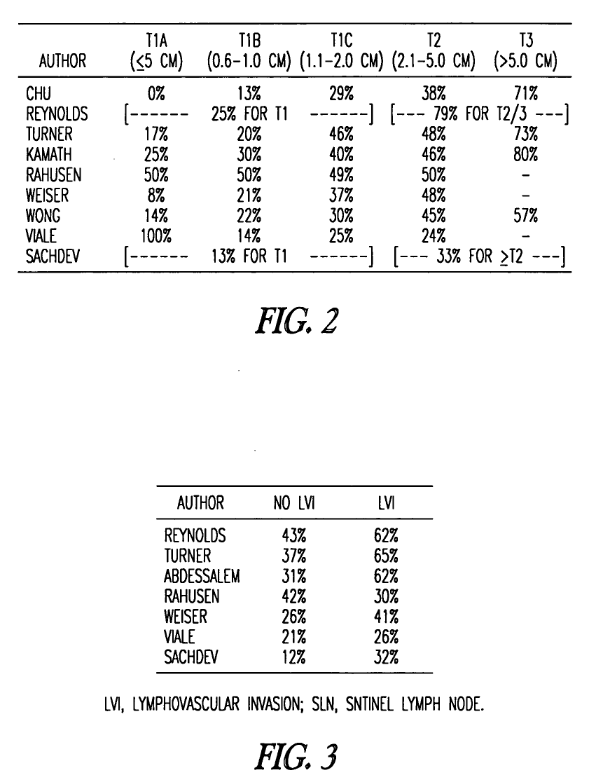

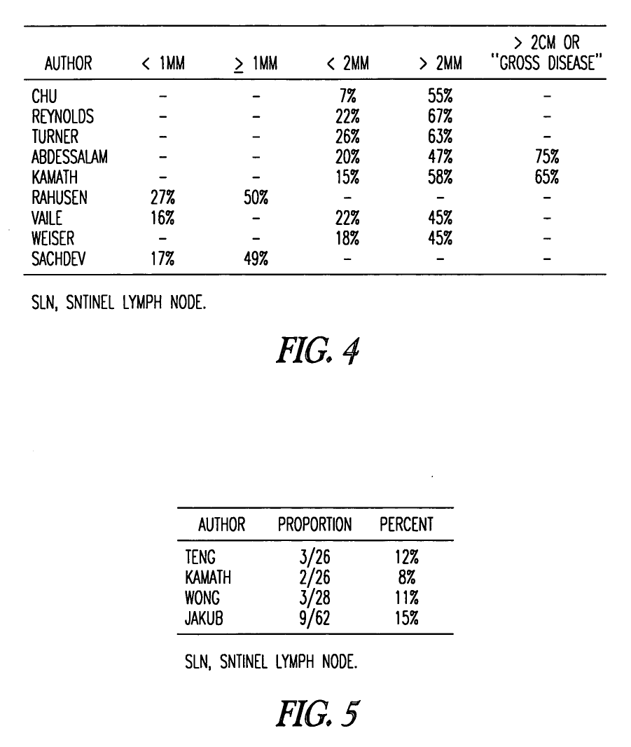

### DETAILED DESCRIPTION OF THE INVENTION

The present invention provides methods, apparatus and nomograms to predict the likelihood of additional nodal metastases in a breast cancer patient with a positive sentinel node biopsy using factors available post-sentinel lymph node biopsy to aid patients considering completion axillary lymph node dissection. In one embodiment, a nomogram predicts the likelihood of additional nodal metastases in breast cancer patients with a positive sentinel node biopsy using patient specific factors to assist the physician and patient in deciding whether or not the patient may benefit from completion axillary lymph node dissection.

One embodiment of the invention is directed to a post-sentinel lymph node biopsy method for predicting the likelihood of additional nodal metastases in breast cancer patients with a positive sentinel node biopsy. The method includes correlating one or more factors, e.g., at least two, three, four, five, six, seven, eight or more factors, determined for each of a plurality of persons previously diagnosed with breast cancer, having a positive sentinel lymph node biopsy and subjected to completion axillary lymph node dissection, with the likelihood of additional nodal metastases for each person of the plurality to generate a functional representation of the correlation. In alternative embodiments, one or more subgroups of any one or more of the following factors may be excluded. The factors include but are not limited to pathologic size of the primary tumor, number of positive sentinel lymph nodes, number of negative sentinel lymph nodes, the method of detection (e.g., frozen, routine, serial HE or IHC), lymphovascular invasion, and/or multifocality, wherein the plurality of persons includes females having undergone sentinel lymph nodes biopsy, having a positive sentinel lymph nodes and completion axillary lymph node dissection. An identical factor or set of factors determined from a breast cancer patient having undergone sentinel lymph node biopsy is employed with the functional representation to predict the likelihood of additional nodal metastases in the breast cancer patient.

In one embodiment, the correlating includes accessing a memory storing the selected set of factors. In another embodiment, the correlating includes generating the functional representation and displaying the functional representation on a display. In one embodiment, the displaying includes transmitting the functional representation from a source. In one embodiment, the correlating is executed by a processor or a virtual computer program. In another embodiment, the correlating includes determining the selected set of factors. In one embodiment, determining includes accessing a memory storing the set of factors from the patient. In another embodiment, the method further comprises transmitting the quantitative probability of additional nodal metastases. In yet another embodiment, the method further comprises displaying the functional representation on a display. In yet another embodiment, the method further comprises inputting the identical set of factors for the patient within an input device. In another embodiment, the method further comprises storing any of the set of factors to a memory or to a database.

In one embodiment, the nomogram is generated with a Cox proportional hazards regression model (Cox, 1972, the disclosure of which is specifically incorporated by reference herein). This method can predict survival-type outcomes using multiple predictor variables. The Cox proportional hazards regression method estimates the probability of reaching a certain end point, such as disease recurrence, over time. In another embodiment, the nomogram may be generated with a neural network model (Rumelhart et al., 1986, the disclosure of which is specifically incorporated by reference herein). This is a non-linear, feed-forward system of layered neurons which backpropagate prediction errors. For instance, an artificial neural network (Dreiseitl et al., 2002, the disclosure of which is specifically incorporated by reference herein) or a Bayesian neural network (Barlow et al., 2001; Hauben et al., 2003, the disclosures of which are specifically incorporated by reference herein) may be employed. In another embodiment, the nomogram may be generated with a recursive partitioning model (Breiman et al., 1984, the disclosure of which is specifically incorporated by reference herein). In yet another embodiment, the nomogram is generated with support vector machine technology (Cristianni et al., 2000; Hastie, 2001, the disclosures of which are specifically incorporated by reference herein). In yet another embodiment, classification and regression trees (CART) can be used (Province et al., 2001; Begg, 1986, the disclosures of which are specifically incorporated by reference herein). Other models known to those skilled in the art may alternatively be used. In one embodiment, the invention includes the use of software that implements Cox regression models or support vector machines to predict the likelihood of additional nodal metastases in breast cancer patients with a positive sentinel node biopsy.

The nomogram may comprise an apparatus for predicting the likelihood of additional nodal metastases in breast cancer patients with a positive sentinel node biopsy. The apparatus comprises a correlation of one or more factors determined for each of a plurality of persons previously diagnosed with breast cancer, having a sentinel lymph node biopsy and having completion axillary lymph node dissection with the incidence of additional nodal metastases for each person of the plurality of persons. The factors include but are not limited to the pathologic size of the primary tumor, the number of positive sentinel lymph node, the number of negative sentinel lymph node, the method of detection (e.g., frozen, routine, serial HE or IHC), nuclear grade, estrogen receptor status, the presence or absence of lymphovascular invasion, and/or the presence or absence of multifocality. The apparatus includes a means for matching an identical set of factors determined from the patient having breast cancer to the correlation to predict the likelihood of additional nodal metastases in the patient with a positive sentinel node biopsy.

The nomogram or functional representation may assume any form, such as a computer program, e.g., in a hand-held device, world-wide-web page, e.g., written in FLASH, or a card, such as a laminated card. Any other suitable representation, picture, depiction or exemplification may be used. The nomogram may comprise a graphic representation and/or may be stored in a database or memory, e.g., a random access memory, read-only memory, disk, virtual memory or processor.

The apparatus comprising a nomogram may further comprise a storage mechanism, wherein the storage mechanism stores the nomogram; an input device that inputs the identical set of factors determined from a patient into the apparatus; and a display mechanism, wherein the display mechanism displays the quantitative likelihood of additional nodal metastases in breast cancer patients with a positive sentinel node biopsy. The storage mechanism may be random access memory, read-only memory, a disk, virtual memory, a database, and a processor. The input device may be a keypad, a keyboard, stored data, a touch screen, a voice activated system, a downloadable program, downloadable data, a digital interface, a hand-held device, or an infra-red signal device. The display mechanism may be a computer monitor, a cathode ray tub (CRT), a digital screen, a light-emitting diode (LED), a liquid crystal display (LCD), an X-ray, a compressed digitized image, a video image, or a hand-held device. The apparatus may further comprise a display that displays the quantitative likelihood of additional nodal metastases in breast cancer patients with a positive sentinel node biopsy, e.g., the display is separated from the processor such that the display receives the quantitative likelihood of additional nodal metastases in breast cancer patients with a positive sentinel node biopsy. The apparatus may further comprise a database, wherein the database stores the correlation of factors and is accessible by the processor. The apparatus may further comprise an input device that inputs the identical set of factors determined from the patient with breast cancer into the apparatus. The input device stores the identical set of factors in a storage mechanism that is accessible by the processor. The apparatus may further comprise a transmission medium for transmitting the selected set of factors. The transmission medium is coupled to the processor and the correlation of factors. The apparatus may further comprise a transmission medium for transmitting the identical set of factors determined from the patient with breast cancer, preferably the transmission medium is coupled to the processor and the correlation of factors. The processor may be a multi-purpose or a dedicated processor. The processor includes an object oriented program having libraries, said libraries storing said correlation of factors.

In one embodiment, the nomogram comprises a graphic representation of a likelihood of additional nodal metastases in breast cancer patients with a positive sentinel node biopsy. The nomogram comprises a substrate or solid support, and a set of indicia on the substrate or solid support, the indicia including one or more of a line for the pathological size, number of positive SLN, number of negative SLN, method of detection, lymphovascular invasion, and multifocality, a total points line and a predictor line, wherein the line for the pathological size, number of positive SLN, number of negative SLN, method of detection (frozen, routine, serial HE or IHC), lymphovascular invasion, and multifocality, each have values on a scale which can be correlated with values on a scale on the points line. The total points line has values on a scale which may be correlated with values on a scale on the predictor line, such that the value of each of the points correlating with the indicia can be added together to yield a total points value, and the total points value correlated with the predictor line to predict the likelihood of additional nodal metastases in breast cancer patients with a positive sentinel node biopsy. The solid support may assume any appropriate form such as, for example, a laminated card. Any other suitable representation, picture, depiction or exemplification may be used.

In addition to assisting the patient and physician in selecting an appropriate course of therapy, the nomograms of the present invention are also useful in clinical trials to identify patients appropriate for a trial, to quantify the expected benefit relative to baseline risk, to verify the effectiveness of randomization, to reduce the sample size requirements, and to facilitate comparisons across studies.

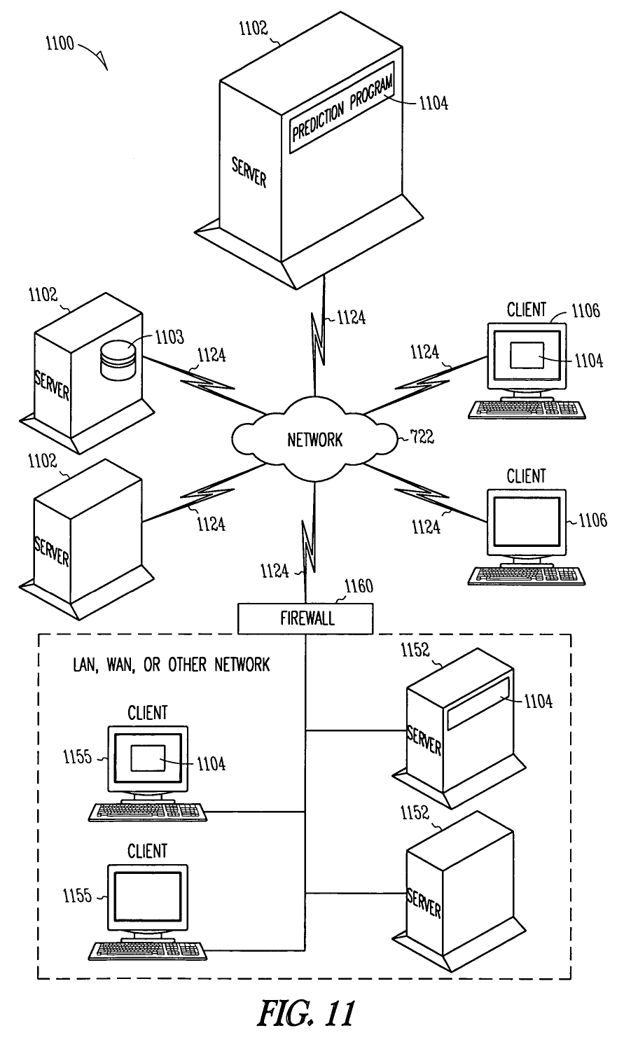

A block diagram of a computer system that executes programming for predicting a prognosis probability is shown in FIG. 10. A general computing device in the form of a computer 1010, may include a processing unit 1002, memory 1004, removable storage 1012, and non-removable storage 1014. Memory 1004 may include volatile memory 1006 and non-volatile memory 1008. Computer 1010 may include—or have access to a computing environment that comprises—a variety of computer-readable media, such as volatile memory 1006 and non-volatile memory 1008, removable storage 1012 and non-removable storage 1014. Computer storage comprises RAM, ROM, EPROM & EEPROM, flash memory or other memory technologies, CD ROM, Digital Versatile Disks (DVD) or other optical disk storage, magnetic cassettes, magnetic tape, magnetic disk storage or other magnetic storage devices, or any other medium capable of storing computer-readable instructions. Computer 1010 may include or have access to a computing environment that comprises input 1016, output 1018, and a communication connection 1020. Input 1016 may include one or several devices such as a keyboard, mouse, touch screen, and stylus. Output 1018 may include one or several devices such as a video display, a printer, an audio output device, a touch stimulation output device, or a screen reading output device. The computer may operate in a networked environment using a communication connection 1020 to connect to one or more remote computers. The remote computer may include a personal computer, server, router, network PC, a peer device or other common network node, or the like. The communication connection 1020 may include a Local Area Network (LAN), a Wide Area Network (WAN) or other networks. The communication connection 1020 may be over a wired network, wireless radio frequency network, or an infrared network. Further, in some embodiments, the network may be a combination of several connection technologies including wired, RF, and/or infrared.

Computer-readable instructions stored on a computer-readable medium are executable by the processing unit 1002 of the computer 1010. A hard drive, CD-ROM, and RAM are some examples of articles including a computer-readable medium. The computer-readable instructions allow computer system 1000 to provide generic access controls in a computer network system having multiple users and servers, wherein communication between the computers includes utilizing TCP/IP, COM, DCOM, XML, Simple Object Access Protocol (SOAP), and Web Services Description Language (WSDL), and other related connection communication protocols and technologies that will be readily apparent to one of skill in the relevant art.

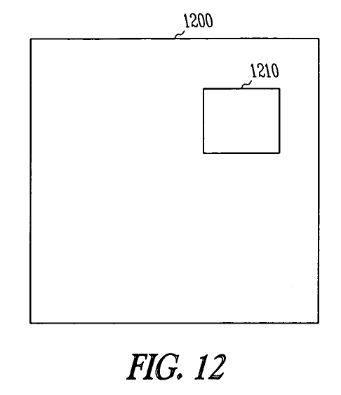

FIG. 11 shows an exemplary embodiment of an information processing system 1100 that provides for transfer of data between multiple devices. This embodiment of system 1100 comprises multiple servers 1102, client work stations 1106, the servers 1102 and client workstations 1106 operatively connected via communication lines 1124 to a network 1122. In one embodiment, network 1122 includes the Internet, or other type of public or private network that allows data transfer. Communication lines 1124 may be any type of communication medium, such as telephone lines, cable, optical fiber, wireless, or any other communication medium that allows data transfer between devices coupled to the network.

In some embodiments, one or more of the servers 1102 hold a prediction program 1104, which is available for download to the other servers 1102 and workstation clients 1106 connected 1124 to the network 1122.

In some other embodiments, prediction program 1104 is executable on a server 1102 wherein the prediction program executes in response to stimulation received from a client 1104 using a Hyper-Text Transfer Protocol (HTTP or HTTPS). In one such embodiment, prediction program 1104 accepts input from a client, executes, and outputs a prognosis prediction in a markup language such as Hyper-Text Markup Language (HTML) or extensible Markup Language (XML).

In some embodiments, system 1100 may be implemented with servers 1102 utilizing one of many available operating systems. Servers 1102 may also include, for example, machine variants such as personal computers, handheld personal digital assistants, RISC processor computers, MIP single and multiprocessor class computers, and other personal, workgroup, and enterprise class servers. Further, servers 1102 may also be implemented with relational database management systems 1103 and application servers. Other servers 1102 may be file servers.

Client workstations 1102 within embodiments of system 1100, may include personal computers, computer terminals, handheld devices, and multifunction mobile phones. Client workstations 1102 include software thereon for performing operations in accordance with stimulation received from a user and signals received from other computing devices on the network 1122. Further, a client workstation 1102 may include a web browser for displaying web pages.

The network 1122 within some embodiments of a system 1100 may include a Local Area Network (LAN), Wide Area Network (WAN), or other similar network 1145 connected 1124 network 1122. Network 1122 may itself be a LAN, WAN, the Internet, or other large scale regional, national, or global network or a combination of several types of networks. Some embodiments of system 1100 include a LAN, WAN, or other similar network 1145 that utilizes one or more servers 1152 and clients 1155 behind a firewall 1160 within the LAN, WAN, or other similar network 1145.

FIG. 12 shows an exemplary embodiment of a machine-readable medium 1200 with operable instructions 1210 thereon for performing the methods described herein on an appropriately configured information processing device. Such devices include in various embodiments personal computers including desktop, laptop, and tablet computers. Some further embodiments include handheld devices utilizing Palm/OS or Windows CE.

The invention will be further described by the following non-limiting examples.

### EXAMPLE I

In an attempt to achieve a more precise prediction for the individual patient than is readily available by using published estimates of risk, a multivariable logistic-regression analysis of a large data set was used to model the association between selected variables and the likelihood of metastases in non-SLN in patients with a positive SLN biopsy. The pathologic size of the primary tumor, the method of detection of the SLN metastasis, as well as several other variables that are readily available and likely related to risk of additional nodal disease, were examined. 702 cases from a large prospective sentinel lymph node database were employed to develop the model, and a nomogram was developed to predict the likelihood of finding additional positive nodes at completion ALND. The model was then tested by prospectively applying it to an additional study group comprising 373 patients. This tool allows greater individualization of a patient's risk estimate by simultaneously taking into account several pertinent characteristics specific to the patient. With a more precise and individualized estimate, both physician and patient are better able to weigh the pros and cons of further axillary dissection.

Materials and Methods

4,790 consecutive cases of SLN biopsy at Memorial Sloan-Kettering Cancer Center (MSKCC) were entered prospectively into the MSKCC Breast Cancer Sentinel Lymph Node Database. The study population was the subset of 1,075 that fulfilled the following criteria: primary invasive breast carcinoma with clinically negative axilla and no prior systemic treatment, successful SLN biopsy in which metastatic disease was identified, and completion ALND with at least 10 nodes examined. There were a total of 140 cases that were excluded because a completion ALND was not performed. Patients meeting selection criteria included the overall study population, which was then divided into two groups: a retrospective group who had undergone SLN biopsy over about 4½ years, and a prospective group undergoing SLN biopsy for the subsequent 1½ years. This project was reviewed and approved by the MSKCC Institutional Review Board.

The technique for SLN biopsy includes the use of both blue dye and radioisotope as described in Cody (2001).

SLN Histopathologic Evaluation

Whenever possible, the SLN was bisected and sectioned at 2- to 3-mm intervals. The nodal tissue was quick frozen in liquid nitrogen and a single, 5-μm-thick hematoxylin and eosin (H&E) stained section was examined intraoperatively (frozen-section analysis). If positive, a complete ALND was done immediately. Following the frozen section, the remaining frozen tissue was fixed in formalin and embedded in paraffin. Another 5-μm-thick H&E-stained section was evaluated as a “frozen section control” (routine histopathology). If this section showed evidence of metastatic disease, no further pathological workup of the SLN was performed. If the routine H&E section remained negative, enhanced pathologic analysis was performed in the following fashion: two pairs of H&E- and cytokeratin IHC-stained sections with a distance of 50 μm between the pairs were prepared from the paraffin block. At one level, the cytokeratin antibody CAM 5.2 (Becton Dickinson Immunocytometry Systems, San Jose, Calif.) was used, while the cytokeratin cocktail AE1:AE3 (Ventana Medical Systems, Inc., Tucson, Ariz.) was applied for the other level. Patients with SLN metastases not detected by frozen-section analysis generally underwent completion ALND at a later date. All additional nodes identified by completion ALND underwent routine H&E analysis of a single section of each node.

Data Analysis

Clinical data collected for each case from the database included age; pathologic size of the invasive carcinoma, defined in centimeters (cm); tumor type (ductal or lobular carcinoma); nuclear grade (I: slight or no variation in size and shape of nucleus; II: moderate variation in size and shape; III: marked variation in size and shape); presence of lymphovascular invasion (presence of a one or more tumor cells in a lymphatic or vascular structure); multifocality of primary tumor (foci of carcinoma separate from primary tumor); estrogen-receptor (ER-) status (negative, <10% of cells staining positive); method of detection of SLN metastases (frozen-section analysis, “Frozen”; routine histopathology, “Routine”; H&E stains of serial sections, “Serial HE”; immunohistochemistry, “IHC”); number of positive SLNs; and number of negative SLNs. Because lobular carcinomas are generally not assigned a nuclear grade, tumor type and nuclear grade were combined into the following four categories: ductal carcinoma, nuclear grade I; ductal carcinoma, nuclear grade II; ductal carcinoma, nuclear grade III; lobular carcinoma.

To allow use of the model by groups that do not routinely perform frozen-section analysis, a second model was developed with only three levels for the method of detection variable: routine histopathology, serial sectioning, and IHC. In this model, a node in which metastatic disease was detected by either frozen-section analysis or routine histopathology was categorized as “routine”. Data on additional variables such as progesterone-receptor status, histologic grade, and AJCC T stage were also collected; however, because these variables are highly correlated with ER-status, nuclear grade, and pathologic size, respectively, it was felt they would not be of substantial benefit to the model. HER-2/neu amplification data were also collected, but were not included because they were incomplete and variable owing to evolving methods of assessment during the years of the study.

A nomogram was developed based on the patients in the retrospective group, and then validated with the patients in the prospective group. In the retrospective population (n=702), multivariable logistic regression was used to analyze the association of each variable with the likelihood of non-SLN metastases, and a nomogram was created with all variables. This model was used in the prospective group (n=373) to predict each individual patient's probability of having positive non-SLNs. The discrimination of the model was measured by using the area under the receiver operating characteristic (ROC) curve. The calibration of the model was assessed graphically. Women were grouped into deciles based on their nomogram predictions. For each decile, the mean nomogram-predicted probability was compared with the proportion of women who actually had positive non-SLNs (actual probability). All analyses were performed using S-Plus software Version 2000 Professional Edition with the Design Library (Mathsoft Data Analysis Products Division, Seattle, Wash.) (Harrell, 2001).

Results

Descriptive characteristics of the study population are listed in Table 1.

TABLE 1

| | | Retrospective | | Prospective | |
|---|---|---|---|---|---|
| | | (n = 702) | | (n = 373) | |
| | | n | % | n | % |
| Age | ≦50 | 290 | 41.3% | 157 | 42.1% |
| | >50 | 412 | 58.7% | 216 | 57.9% |
| Pathologic size (cm) | ≦0.5 | 33 | 4.7% | 13 | 3.5% |
| | 0.6-1.0 | 122 | 17.4% | 49 | 13.1% |
| | 1.1-2.0 | 312 | 44.4% | 166 | 44.5% |
| | 2.1-3.0 | 154 | 21.9% | 93 | 24.9% |
| | 3.1-5.0 | 65 | 9.3% | 41 | 11.0% |
| | ≧5.1 | 16 | 2.3% | 11 | 2.9% |
| Tumor type and nuclear | Ductal, I | 22 | 3.1% | 11 | 2.9% |
| grade | Ductal, II | 321 | 45.7% | 175 | 46.9% |
| | Ductal, III | 275 | 39.2% | 129 | 34.6% |
| | Lobular | 84 | 12.0% | 58 | 15.5% |
| Lymphovascular invasion | No | 418 | 59.5% | 219 | 58.7% |
| | Yes | 284 | 40.5% | 154 | 41.3% |
| Multifocal | No | 505 | 71.9% | 241 | 64.6% |
| | Yes | 197 | 28.1% | 132 | 35.4% |
| Estrogen-receptor status | Negative | 135 | 19.2% | 83 | 22.3% |
| | Positive | 567 | 80.8% | 290 | 77.7% |
| Method of detection | IHC only | 63 | 9.0% | 18 | 4.8% |
| | Serial H&E | 78 | 11.1% | 40 | 10.7% |
| | Routine | 65 | 9.3% | 23 | 6.2% |
| | Frozen | 463 | 66.0% | 273 | 73.2% |
| | Frozen not | 33 | 4.7% | 19 | 5.1% |
| | done | | | | |
| Number of positive SLN | 1 | 488 | 69.5% | 265 | 71% |
| | 2 | 161 | 22.9% | 75 | 20.1% |
| | 3 | 35 | 5.0% | 21 | 5.6% |
| | 4 | 12 | 1.7% | 8 | 2.1% |
| | 5 | 3 | 0.4% | 3 | 0.8% |
| | 6 | 1 | 0.1% | 0 | 0% |
| | 7 | 2 | 0.3% | 0 | 0% |
| | ≧8 | 0 | 0% | 1 | 0.3% |
| Number of negative SLN | 0 | 271 | 38.6% | 132 | 35.4% |
| | 1 | 183 | 26.1% | 79 | 21.2% |
| | 2 | 102 | 14.5% | 72 | 19.3% |
| | 3 | 68 | 9.7% | 41 | 11.0% |
| | 4 | 34 | 4.8% | 22 | 5.9% |
| | 5 | 16 | 2.3% | 7 | 1.9% |
| | 6 | 6 | 0.9% | 10 | 2.7% |
| | 7 | 8 | 1.1% | 2 | 0.5% |
| | ≧8 | 14 | 2.0% | 8 | 2.1% |

SLN, sentinel lymph node.

Table 2 shows the incidence of additional, non-SLN metastases for retrospective, prospective, and total patient populations by primary and SLN pathologic characteristics.

TABLE 2

| | | Retrospective | | Prospective | | Entire population | |
|---|---|---|---|---|---|---|---|
| | | (n = 702) | | (n = 373) | | (n = 1075) | |
| | | Proportion | % | Proportion | % | Proportion | % |
| Age | ≦50 | 114/290 | 39% | 64/157 | 41% | 178/447 | 40% |
| | >50 | 150/412 | 36% | 90/216 | 42% | 240/628 | 38% |
| Pathologic size (cm) | ≦0.5 | 8/33 | 24% | 1/13 | 8% | 9/46 | 20% |
| | 0.6-1.0 | 32/122 | 26% | 13/49 | 27% | 45/171 | 26% |
| | 1.1-2.0 | 111/312 | 36% | 60/166 | 36% | 171/478 | 36% |
| | 2.1-3.0 | 62/154 | 40% | 44/93 | 47% | 106/247 | 43% |
| | 3.1-5.0 | 37/65 | 57% | 26/41 | 63% | 63/106 | 59% |
| | ≧5.1 | 14/16 | 88% | 10/11 | 91% | 24/27 | 89% |
| Tumor type and nuclear | Ductal, I | 6/22 | 27% | 3/11 | 27% | 9/33 | 27% |
| grade | Ductal, II | 100/321 | 31% | 57/175 | 33% | 157/496 | 32% |
| | Ductal, III | 121/275 | 44% | 67/129 | 52% | 188/404 | 47% |
| | Lobular | 37/84 | 44% | 27/58 | 47% | 64/142 | 45% |
| Lymphovascular | No | 125/418 | 30% | 71/219 | 32% | 196/637 | 31% |
| invasion | Yes | 139/284 | 49% | 83/154 | 54% | 222/438 | 51% |
| Multifocality | No | 176/505 | 35% | 89/241 | 37% | 265/746 | 36% |
| | Yes | 88/197 | 45% | 65/132 | 49% | 153/329 | 47% |
| Estrogen-receptor status | Negative | 50/135 | 37% | 37/83 | 45% | 87/218 | 40% |
| | Positive | 214/567 | 38% | 117/290 | 40% | 331/857 | 39% |
| Method of detection | IHC only | 6/63 | 10% | 4/18 | 22% | 10/81 | 12% |
| | Serial H&E | 12/78 | 15% | 3/40 | 8% | 15/118 | 13% |
| | Routine | 13/65 | 20% | 6/23 | 26% | 19/88 | 22% |
| | Frozen | 221/463 | 48% | 128/273 | 47% | 349/736 | 47% |
| | Frozen not | 12/33 | 36% | 13/19 | 68% | 25/52 | 48% |
| | done | | | | | | |
| Number of positive SLN | 1 | 155/488 | 32% | 97/265 | 37% | 252/753 | 33% |
| | 2 | 72/161 | 45% | 34/75 | 45% | 106/236 | 45% |
| | 3 | 23/35 | 66% | 15/21 | 71% | 38/56 | 68% |
| | 4 | 9/12 | 75% | 5/8 | 63% | 14/20 | 70% |
| | 5 | 2/3 | 67% | 2/3 | 67% | 4/6 | 67% |
| | 6 | 1/1 | 100% | 0/0 | — | 1/1 | 100% |
| | 7 | 2/2 | 100% | 0/0 | — | 2/2 | 100% |
| | ≧8 | 0/0 | — | 1/1 | 100% | 1/1 | 100% |
| Number of negative SLN | 0 | 134/271 | 49% | 83/132 | 63% | 217/403 | 54% |
| | 1 | 67/183 | 37% | 25/79 | 32% | 92/262 | 35% |
| | 2 | 27/102 | 27% | 18/72 | 25% | 45/174 | 26% |
| | 3 | 18/68 | 26% | 14/41 | 34% | 32/109 | 29% |
| | 4 | 11/34 | 32% | 10/22 | 45% | 21/56 | 38% |
| | 5 | 3/16 | 19% | 0/7 | 0% | 3/23 | 13% |
| | 6 | 1/6 | 17% | 3/10 | 30% | 4/16 | 25% |
| | 7 | 2/8 | 25% | 1/2 | 50% | 3/10 | 30% |
| | ≧8 | 1/14 | 7% | 0/8 | 0% | 1/22 | 5% |

SLN, sentinel lymph node.

On multivariable logistic-regression analysis, pathologic size, lymphovascular invasion, method of detection, number of positive SLNs, and number of negative SLNs, were each associated with the likelihood of additional, non-SLN metastases (P<0.05 for each). Multifocality was of borderline significance, and neither tumor type and nuclear grade nor ER status had a statistically significant association with the likelihood of non-SLN metastases (Tables 3 and 4).

TABLE 3

| Variables | P value |
|---|---|
| Pathology size | 0.001 |
| Tumor type and nuclear grade | 0.7 |
| Ductal, nuclear grade I vs II | 1.0 |
| Ductal, nuclear grade I vs III | 0.7 |
| Ductal nuclear grade I vs Lobular | 0.8 |
| Lymphovascular invasion | 0.003 |
| Multifocality | 0.06 |
| Estrogen-receptor status | 0.08 |
| Method of detection | <0.001 |
| Frozen vs IHC | <0.001 |
| Frozen vs Serial HE | <0.001 |
| Frozen vs Routine | <0.001 |
| Number of positive SLN | <0.001 |
| Number of negative SLN | <0.001 |

HE, hematoxylin and eosin;
IHC, immunohistochemistry,
SLN, sentinel lymph node.

TABLE 4

| Variables | P value |
|---|---|
| Pathology size | 0.0006 |
| Tumor type and nuclear grade | 0.4 |
| Ductal, nuclear grade I vs II | 0.8 |
| Ductal, nuclear grade I vs III | 0.4 |
| Ductal nuclear grade I vs Lobular | 0.6 |
| Lymphovascular invasion | 0.003 |
| Multifocality | 0.02 |
| Estrogen-receptor status | 0.16 |
| Method of detection (no frozen section available) | <0.0001 |
| Routine vs IHC | 0.0001 |
| Routine vs Serial HE | <0.001 |
| Number of positive SLN | 0.0001 |
| Number of negative SLN | <0.0001 |

HE, hematoxylin and eosin;
IHC, immunohistochemistry,
SLN, sentinel lymph node.

Age was not included in the final nomograms because its effect was too small to be seen on the nomograms.

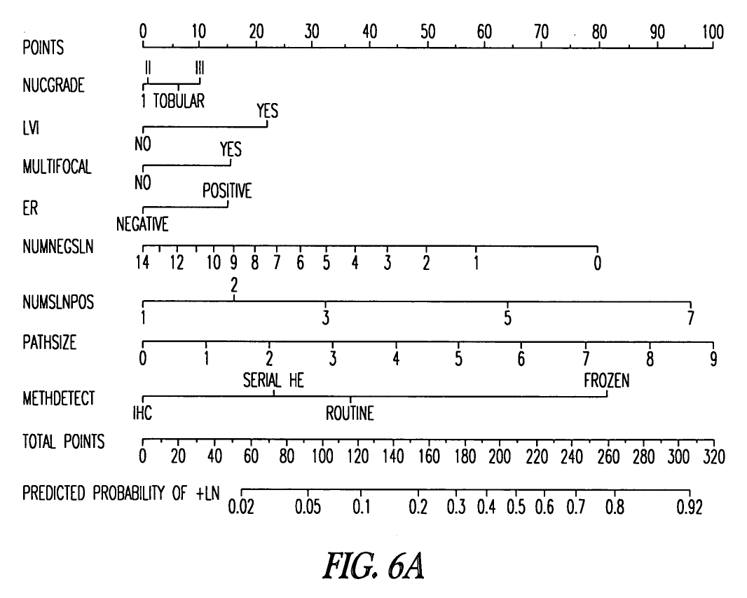

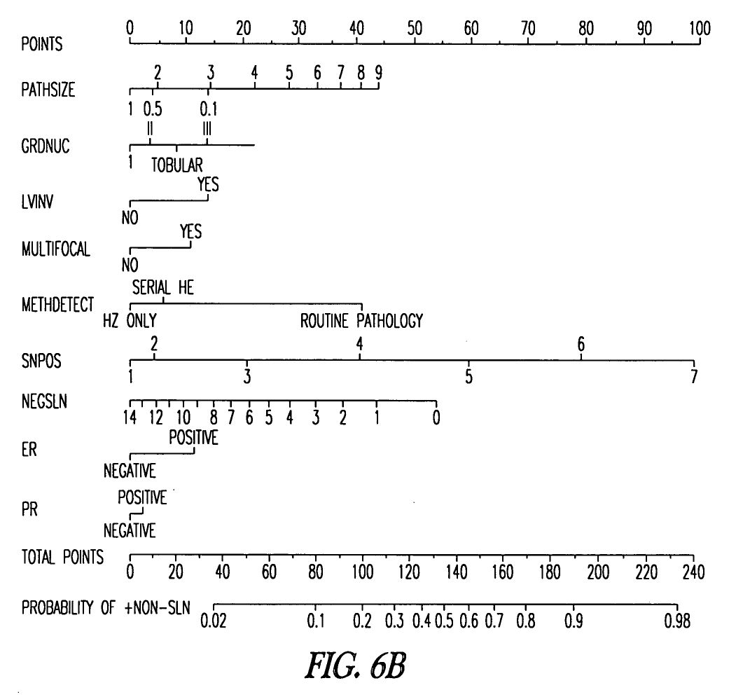

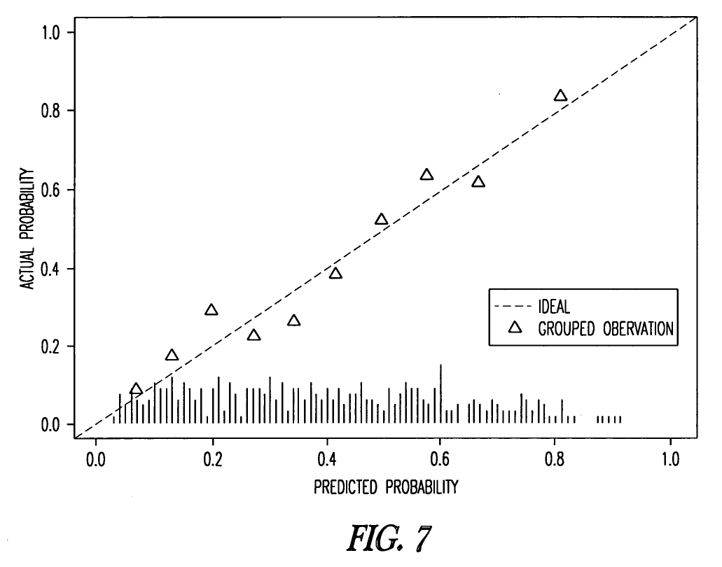

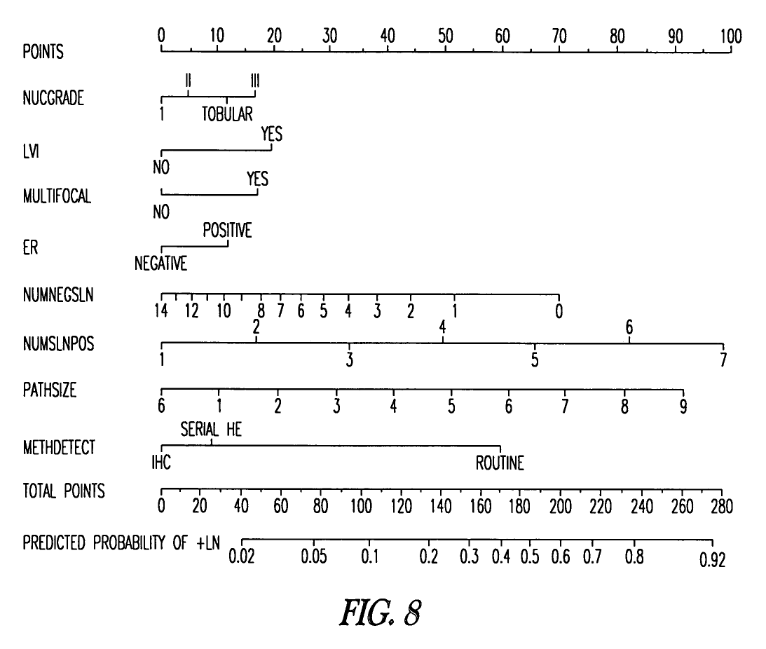

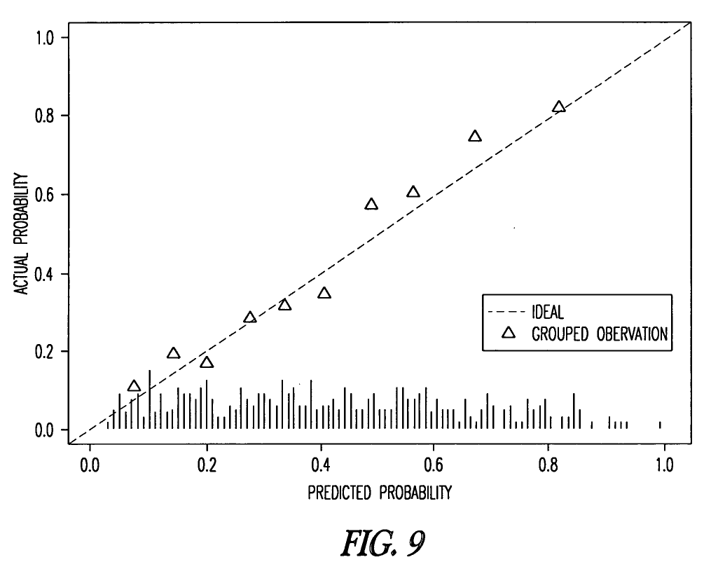

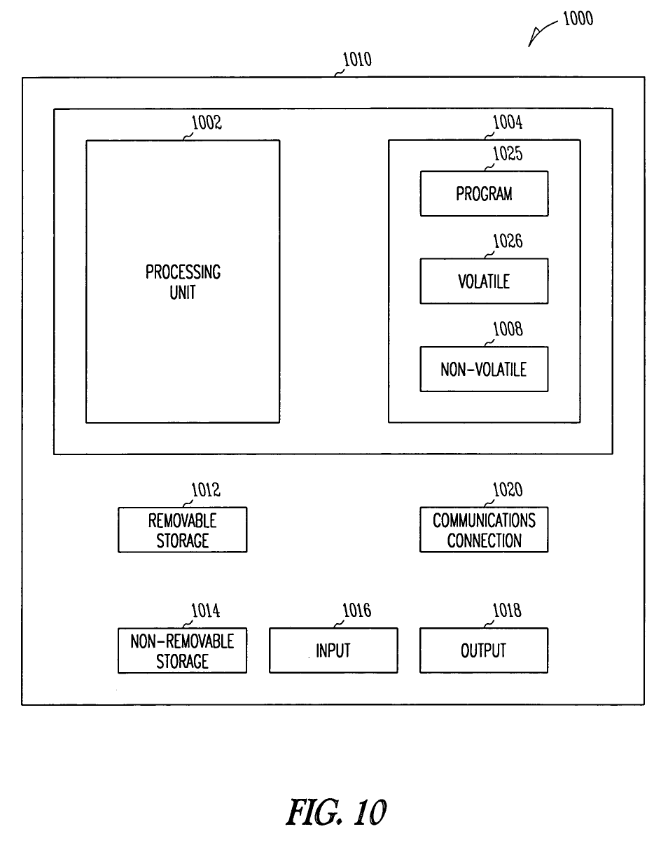

A nomogram based on this model and developed in the retrospective population (n=702) is shown in FIG. 6. The overall predictive accuracy of a model incorporating the eight variables, as measured by the bootstrap corrected ROC curve, was 0.76. To address the calibration accuracy of the nomogram (i.e., the absolute error of its prediction), additional bootstrapping was conducted and the probabilities predicted by the nomogram plotted against the corresponding observed proportions in the prospective population (n=373) (FIG. 7). The area under the ROC curve for the model applied to the prospective population is 0.77. For those users who do not routinely perform frozen-section analysis on the SLN, a separate analysis without frozen-section information was performed and is illustrated in the nomogram shown in FIG. 8. The ROC of this version of the nomogram is 0.75 in the retrospective population and 0.78 in the prospective population. The corresponding calibration curve is depicted in FIG. 9.

Using the Nomogram

Each version of the nomogram consists of 11 rows. The first row (POINTS) is the point assignment for each variable. Rows 2-9 represent the variables included in the model. For an individual patient, each variable is assigned a point value (uppermost scale “POINTS”) based on the histopathologic characteristics. To determine the point assignment, a vertical line is made between the appropriate variable value and the POINTS line. For example, a pathologic size of 1 cm (PATHSIZE, 2) confers about 10 points.

The assigned points for all eight variables are summed, and the total is found in row 10 (TOTAL POINTS). Once the total is located in row 10, a vertical line is made between it and the corresponding value in the final row, Row 11 (Predicted Probability of +LN). The version of the nomogram in FIG. 6 is for use when information on frozen-section analysis is available; that in FIG. 8 is for those cases where frozen-section information is not available.

In addition to the graphical nomograms, to facilitate ease of use in the clinical setting, a personal digital assistant (PDA)-compatible application for use on hand-held Palm™-type devices is provided at www.mskcc.org/nomograms.

Discussion

With the adoption of SLN biopsy, a new clinical conundrum has become commonplace: should a completion ALND be done for a patient with a positive SLN biopsy? This question is particularly difficult in patients with micrometastatic disease, disease which was undetectable in the era prior to SLN biopsy. Other investigators have attempted to address this question, and have identified risk factors for the presence of additional, non-SLN disease, but all such attempts are limited by the practical difficulty of simultaneously including several variables in the risk estimate. Here, simultaneously using several variables in a large population, nomograms were developed to predict the likelihood of additional nodal metastases after a positive SLN biopsy. The nomograms were prospectively tested and shown to perform well in the prospective population.

The nomograms utilize available clinical information, and allow quick calculation. This approach may allow identification of extremely low-risk individuals in whom the risks associated with completion ALND are judged to outweigh the benefits. On the other hand, the nomogram may allow identification of women at sufficient risk of additional nodal disease that they and their surgeon elect to proceed with completion ALND even though clinical “guesstimates” would suggest that they are at “low risk.”

The nomograms provide risk estimates that are judged on an individual basis. A woman with a 1.8-cm, ER-positive, high-nuclear-grade ductal carcinoma with no LVI, who has a single IHC-positive SLN might be considered to be “low risk.” The nomogram suggests that she has a 12% risk of having non-SLN metastases. Should she undergo completion ALND? Given this scenario, some will judge that a 12% risk of additional, non-SLN metastases justifies further ALND, others will not. The nomogram itself makes no actual treatment recommendations.

The nodes retrieved at completion ALND were examined by routine pathologic analysis only. Other investigators (Turner et al., 2000; Chu et al., 1999) have shown that if non-SLNs are examined with serial sectioning and IHC, a higher proportion of patients with additional, non-SLN disease at completion ALND are identified.

Moreover, the clinical relevance of resecting additional nodal disease (even that detected by routine analysis) remains unknown. While some argue that surgical removal of subclinical nodal disease is associated with a small, but non-zero, survival benefit, others argue that current adjuvant systemic therapy and radiation therapy would likely treat the majority of patients adequately. This study does not address this issue, but rather provides accurate and individualized estimates of the likelihood there will be additional disease at completion ALND. The American College of Surgeons Oncology Group Protocol Z0011 (ACOSOG Z0011), currently under way and randomizing women with a positive SLN to ALND or no, is designed to address this question directly.

In addition, the prognostic significance of micrometastatic nodal disease is a subject of debate. In a 1997 review of the published literature, Dowlatshahi et al. (1997) concluded that all but one of the large (N≧147) and long-term (≧6 yrs) studies demonstrated a statistically significant decrement in survival associated with micrometastatic disease. Tan et al. (2002) recently re-examined all axillary nodes from 373 patients treated in the 1970s who were deemed to be node-negative by routine histopathologic analysis. Nodes were examined by serial sectioning and IHC, and the presence of any detectable micrometastatic disease was associated with worse disease-free and overall survival.

The AJCC Cancer Staging Manual, 6th edition, now includes size of metastasis as an important determination of stage (and, therefore, of prognosis). However, it can be difficult to assign a size to many cases because of the difference in pattern of distribution of malignant cells within the node. For example, some nodes may have scattered single cells or multiple small clusters of cells. How should these be measured? Ideally, an accurate estimate of volume could be assigned to each SLN metastasis. However, this is extremely time-consuming and somewhat impractical.

Nevertheless, it is clear that IHC is more sensitive than H&E in detecting micrometastases, that routine H&E analysis is more sensitive than frozen-section analysis, and that there is a correlation between method of detection and volume of disease. Kamath et al. (2001) and Rahusen et al. (2001) have demonstrated quantitatively that method of detection is correlated with measured size of the SLN metastasis. Therefore, in order to have a consistent, practical, and reproducible methodology of estimation, the method of detection of the nodal metastasis was used. This provides a general estimate of the amount of nodal disease, and allows grouping into four distinct groups.

Some of the patients in the present study, especially those with a perceived low risk of additional, non-SLN metastases, did not have a completion ALND and therefore were not included in the model. However, as demonstrated in the histograms (FIGS. 7 and 9), the patients in the prospective population are distributed quite evenly across the range of predicted risk. Furthermore, as demonstrated on the calibration curves, the models do predict well in the prospective population, even for those in the lowest decile of predicted risk.

The models described herein represent a significant improvement over estimates based on one or two variables in smaller populations. A large, prospective database was used to develop the models, and their validity proven by testing them prospectively on a subsequent population. The calibration errors of the models are small (see FIGS. 7 and 9), generally less than 10% across the spectrum of predictors. Other investigators have shown that removing statistically insignificant predictors actually worsens predictive ability of the model (Harrell et al., 1996). Here, all statistically significant variables were incorporated, as well as other clinically available and relevant variables, to provide improved prediction capability. Nomograms provide improved predictive ability when compared with the crude counting of risk factors, and in addition, nomograms usually outperform clinical judgment, based on numerous studies conducted in other areas of medicine (Ross et al., 2002).

With the important clinical question of whether to perform a completion ALND in a patient with a positive SLN biopsy arising more and more frequently, the present nomograms provide an easy-to-use tool with which to simultaneously incorporate several important variables into the estimate of risk of additional, non-SLN metastases. These nomograms provide a risk estimate that can help in weighing the pros and cons of completion ALND for an individual patient with SLN metastases.

### EXAMPLE II

Methods

Construction of the nomogram is described in Example I. In brief, 702 cases of primary breast cancer in which the SLN was positive for metastasis were identified from a prospectively collected SLN database. Using primary tumor and SLN metastasis characteristics, a multivariate model was created to predict the likelihood of additional, non-SLN metastases being found at completion ALND. The model was subsequently applied prospectively to an additional 373 patients (validation population), and found to accurately predict the likelihood of residual disease (area under the receiver operating characteristic curve=0.77).

For experiment I, 33 women were selected at random from the validation population used to confirm the original nomogram. The characteristics of these women were supplied to 17 participating clinicians for their prediction (Table 5). Clinicians were asked, for each patient, “If 100 women with these characteristics were to have a positive sentinel node and then receive a full axillary dissection, how many of them would you expect to have one or more positive non-sentinel lymph nodes?” Clinicians included specialists in breast cancer who attended a weekly multidisciplinary breast conference. Individual specialties included: surgeons, medical oncologists, radiation oncologists, radiologists, and pathologists. These clinicians were unfamiliar with the nomogram and had not yet incorporated it into their clinical practice at the time they participated in this experiment.

TABLE 5
If 100 women with these characteristics were to have a positive sentinel node and then receive a full ax dissection,
how many of them would you expect to have one or more positive non-sentinel nodes? Please write down a number
from 0-100 for each case. Please write in both columns, tear on vertical line and give us the smaller list.

| | | | | | | | | | | | | # | | | |
|---|---|---|---|---|---|---|---|---|---|---|---|---|---|---|---|
| | | | | | | | | | | | | women | | Attending or Fellow | |
| | | | | | | | | | | | | with pos | | Service | |
| | | | | | | | HOW | NUM | NUM | | | ALND out | | | # women with |
| | | | PATH | NUC | | MULTI | SLN + | SLN | SLN | | | of 100 | | | pos ALND out |
| Case | Age | TLOC | SIZE | GRADE | LVI | FOCAL | DETECT | POS | NEG | ER | PR | women | TEAR | Case | of 100 women |
| 1 | 50 | Central | 3 | II | No | Yes | IHC | 1 | 0 | Neg | Neg | | | 1 | |
| 6 | 62 | UIQ | 2.5 | II | No | No | Serial HE | 1 | 2 | Pos | Pos | | | 6 | |
| 9 | 34 | UOQ | 0.6 | III | No | No | IHC | 1 | 0 | Pos | Neg | | | 9 | |
| 14 | 50 | UOQ | 1.5 | Lobular | No | Yes | Routine | 1 | 0 | Pos | Pos | | | 14 | |
| 39 | 61 | UOQ | 0.9 | II | Yes | Yes | Routine | 1 | 1 | Pos | Pos | | | 39 | |
| 50 | 28 | UIQ | 1.2 | II | Yes | No | Serial HE | 1 | 2 | Neg | Neg | | | 50 | |
| 63 | 67 | UOQ | 2.2 | II | Yes | No | Routine | 1 | 3 | Pos | Pos | | | 63 | |
| 83 | 38 | LOQ | 3.5 | III | No | No | Serial HE | 1 | 2 | Pos | Pos | | | 83 | |
| 123 | 53 | UOQ | 0.5 | II | No | No | Routine | 2 | 3 | Pos | Neg | | | 123 | |
| 129 | 67 | UIQ | 1.8 | Lobular | Yes | Yes | IHC | 1 | 2 | Pos | Pos | | | 129 | |
| 140 | 47 | UOQ | 1.8 | II | Yes | No | Serial HE | 2 | 2 | Pos | Pos | | | 140 | |
| 160 | 33 | UOQ | 2.4 | III | Yes | No | IHC | 1 | 2 | Neg | Neg | | | 160 | |
| 162 | 54 | UIQ | 2.5 | II | Yes | No | Routine | 1 | 2 | Neg | Neg | | | 162 | |
| 170 | 55 | UOQ | 0.4 | II | No | Yes | Serial HE | 1 | 6 | Pos | Neg | | | 170 | |
| 184 | 55 | LIQ | 1.3 | II | No | No | IHC | 1 | 1 | Neg | Pos | | | 184 | |
| 192 | 52 | LOQ | 3.6 | III | No | No | Routine | 1 | 3 | Pos | Pos | | | 192 | |
| 194 | 35 | UOQ | 1.2 | II | No | No | Serial HE | 1 | 2 | Pos | Pos | | | 194 | |
| 203 | 80 | UOQ | 1.5 | Lobular | No | No | Serial HE | 1 | 0 | Pos | Neg | | | 203 | |
| 224 | 29 | UOQ | 2 | III | Yes | No | IHC | 1 | 1 | Pos | Pos | | | 224 | |
| 228 | 53 | UOQ | 4 | Lobular | No | Yes | Routine | 3 | 2 | Pos | Neg | | | 228 | |
| 232 | 45 | UOQ | 1.2 | II | No | Yes | Serial HE | 2 | 8 | Neg | Pos | | | 232 | |
| 236 | 49 | LOQ | 1 | I | No | No | Serial HE | 1 | 2 | Pos | Pos | | | 236 | |
| 242 | 46 | LOQ | 1.4 | III | No | No | Routine | 3 | 2 | Pos | Pos | | | 242 | |
| 269 | 44 | Central | 2.2 | II | No | No | Routine | 1 | 1 | Pos | Neg | | | 269 | |
| 292 | 71 | Central | 3.5 | Lobular | No | Yes | IHC | 1 | 0 | Pos | Neg | | | 292 | |
| 306 | 58 | UOQ | 1.7 | Lobular | No | Yes | Serial HE | 1 | 3 | Pos | Neg | | | 306 | |
| 318 | 84 | UOQ | 1.4 | Lobular | No | Yes | Routine | 1 | 2 | Pos | Neg | | | 318 | |
| 324 | 40 | LIQ | 2 | III | No | No | Serial HE | 1 | 6 | Neg | Neg | | | 324 | |
| 335 | 52 | LOQ | 1.5 | Lobular | No | No | Serial HE | 1 | 4 | Pos | Neg | | | 335 | |
| 342 | 59 | UOQ | 1.3 | II | No | No | Serial HE | 1 | 1 | Pos | Pos | | | 342 | |
| 348 | 60 | LOQ | 1.5 | II | No | No | IHC | 1 | 2 | Pos | Neg | | | 348 | |
| 353 | 44 | UOQ | 0.65 | Lobular | No | No | Serial HE | 2 | 2 | Pos | Pos | | | 353 | |
| 362 | 52 | UOQ | 0.95 | II | No | No | Serial HE | 1 | 0 | Pos | Pos | | | 362 | |

Subsequently, in experiment II, clinicians were presented with tumor and patient characteristics from 8 patients in the validation set. Clinicians were specifically asked, “Would you perform a completion axillary dissection on this patient?” After they answered, they were then presented the results of the nomogram prediction for residual axillary disease in that patient. Clinicians were then asked again, “Would you perform a completion axillary dissection?”

Twenty-four clinicians participated in experiment II. This study was conducted during a multidisciplinary breast cancer conference. The room was equipped with audience participation software and each participant was given a handheld device and responses were recorded for each device. Unfortunately, not all participants responded to all questions. There were 187 responses to 8 questions, which resulted in a 97.4% response rate.

For experiment I, the clinician and nomogram predictions were evaluated by calculating the area under the receiver operating characteristic curve (ROC). The ROC curve is a visual representation of the tradeoff between sensitivity and specificity of a diagnostic test. This curve describes the inherent predictive ability of the test. Each point along the curve corresponds to the sensitivity and specificity for that test threshold. In the present study, the curve describes the sensitivity and specificity of the nomogram at different levels of likelihood of residual disease.

The area under the ROC curve (AUC) is a value measurement that allows the comparison of the predictive ability of two tests. If the AUC is 0.5, then the curve is approximately a straight line, and the test is no better than a flip of the coin in predicting the desired outcome. However, if the AUC is 1.0, the test is perfect, and correctly identifies all the true positives and true negatives.

To compare the accuracy of nomogram and clinician predictions of lymph node positivity, an ROC curve approach was employed. Specifically, the parameters of the ROC curves were estimated using a latent-variable binormal model (Temple et al., 2002). A random effects term was added to account for the fact that each patient was evaluated several times by different physicians. Model estimates were obtained using restricted maximum likelihood with SAS PROC MIXED (SAS/STAT User Manual Version 9, 2003) and the accuracy of nomogram and clinician predictions were compared using a likelihood ratio test.

For experiment II, a mixed model was used, this time to evaluate whether a statistically significant shift in clinician judgment had occurred upon viewing the nomogram predictions.

Results

In experiment I, 17 clinicians made predictions for 33 patients. The nomogram predicted more accurately (AUC=0.72) than the clinicians (AUC=0.54) as a group (P<0.01). One clinician outperformed the nomogram while the remaining 16 made predictions that were inferior to those made by the nomogram.

In experiment II, 24 clinicians responded to 8 questions, which resulted in 187 responses, 97% response rate. Clinicians rarely changed their surgical decision after being presented with the nomogram prediction of non-SLN metastases (Table 6). Ninety percent of responses represented no change in surgical plan (168/187). Among those recommendations for not proceeding with completion ALND, half (7/14) were changed after being presented with the nomogram prediction. Among recommendations for completion ALND, only 7% (12/173) were changed to “no ALND” after presentation of the nomogram prediction.

TABLE 6
Clinicians responses to whether they would recommend a completion
axillary lymph node dissection after a positive sentinel lymph node biopsy.
(n = 187 responses)

| | Clinician would | Clinician would |
|---|---|---|
| | recommend ALND | recommend no ALND |
| | after nomogram | after nomogram |
| | prediction | prediction |
| Clinician would | 161 | 12 |
| recommend ALND before | | |
| nomogram prediction | | |
| Clinician would | 7 | 7 |
| recommend no ALND | | |
| before nomogram | | |
| prediction | | |

Discussion

Outside of a clinical trial, completion ALND after a positive sentinel lymph node biopsy is recommended. However, for patients in whom the perceived risk of residual disease is low, some patients and clinicians feel that the benefit of axillary dissection is outweighed by its risks, and choose not to have a completion ALND. For such patients, the nomogram was developed to provide an accurate risk estimate that can help in weighing the pros and cons of completion ALND.

Uncertainties in medical decision-making are plentiful, including inaccuracies in diagnosis, uncertainties in the natural progression of disease, and variations with regards to the effect of treatment in an individual patient. In discussing the risks and benefits of performing a completion ALND after a positive SLN biopsy, the physician must process abundant data to arrive at their best prediction of the likelihood of finding residual disease. As demonstrated herein, when presented with similar input data to answer this question, a statistical model (nomogram) predicted more accurately than clinical experts the status of the axilla after a positive SLN biopsy.

This study supports a previous finding that nomogram models outperform human experts (Ross et al., 2001). Humans are filled with inherent biases that make predicting outcomes difficult. Clinicians are plagued by recall bias, remembering the unique patient rather than the routine. Control bias occurs when outcomes are predicted that one wants to come true. Practically, clinicians utilize simple rules to stratify patients rather than a continuous regression analysis (Ross et al., 2001). For example, a clinician heuristic might be that tumors over 5 cm in size are “high risk” for nodal metastases rather than utilize size as a continuous variable. Therefore, it is not surprising that the nomogram did perform better than clinical experts. The nomogram is a predictive instrument that can accurately weigh multiple individual variables simultaneously and without bias.

Does this mean that the nomogram should replace the clinical expert in making the decision about completion ALND after a positive SLN biopsy? Of course not, but the nomogram could be added as a tool to the decision making process in providing the clinician with a numerical estimate to help both the clinician and patient weigh the pros and cons in making this decision. There is no inherent ability of a nomogram to perform risk/benefit analyses and therefore it can not replace clinical judgment.

Outside of a clinical trial, completion ALND is recommended after a positive sentinel lymph node biopsy. However, some clinicians and patients may feel that the risk of residual disease is low and that the morbidity of ALND high and therefore may forgo dissection based on this information. The nomogram provides such patients an accurate estimate of the likelihood of residual disease and thereby allow an informed decision.

In experiment II indicates that the nomogram was unable to change physicians' behavior. It is possible that the clinicians were unfamiliar with the nomogram as a prediction tool and that with increased use would be more comfortable relying on the results to be part of the decision making process. Alternatively, reported clinical decisions may not be reliable outside of the clinic and therefore only with a patient present could a true estimate of change in behavior be made. However, the most likely explanation is that clinicians believe that the standard of care is completion ALND after a positive SLNB, and only rarely consider not recommending a completion ALND (14/187=7%). It is informative that of these 14 responses indicating a preference for not doing completion ALND, fully half (7/14=50%) changed their minds to recommend completion ALND after hearing the nomogram estimate of likelihood of residual disease. Conversely, of the 173 recommendations for ALND, only 12 (12/173=7%) were changed to recommend no ALND after hearing the nomogram estimates.

Interestingly, in two scenarios, the decision whether or not to dissect the axilla was different, even though the risk of residual disease was the same. A post-menopausal woman with a 9% risk of residual disease was presented and two clinicians changed their behavior to not perform completion ALND; presumably the nomogram prediction was lower than what the clinician had anticipated. Conversely, a 38-year-old woman with an 8% risk of residual disease was presented and four clinicians changed their response to dissect the axilla—presumably the nomogram prediction was higher than they had predicted. Clearly, age plays a role in making the decision about returning to the operating room. However, in constructing the nomogram, age had no predictive role in determining likelihood of non-SLN metastases. A patient's age is seemingly associated with the amount of risk clinicians are willing to assume.

The question of estimating the risk of non-SLN metastases in the axilla after a positive SLN biopsy is an important one that clinicians are facing more frequently. Accurate estimates of this likelihood may improve risk stratification in future clinical trials of the utility of completion ALND. Further, accurate estimates of risk are essential for an informed discussion with patients regarding the pros and cons of completion ALND in the setting of a positive SLN biopsy. Nomogram predictions appear to be substantially more accurate than clinical predictions, and therefore clinicians can improve their predictive ability by using the nomogram to predict the likelihood of additional non-SLN metastases in a woman with a positive SLN biopsy who is considering not pursuing completion ALND.

### REFERENCES

Abdessalam et al., Am J Surg 2001;182:316.
Albertini et al., JAMA 1996;276:1818.
American Joint Committee on Cancer. Breast. AJCC Cancer Staging Manual, 6^{th} edition. New York: Springer. 2002:221.
Barlow et al., Network 2001;12:241.
Begg, Crit Rev Med Inform 1986; 1:1
Breiman et al., Classification and Regression Trees. Monterey, Calif., Wadsworth and Brooks/Cole (1984).
Cabanes et al., Lancet 1992;339:1245.
Cady, J Surg Oncol 1997;66:7.
Carter et al., Cancer 1989;63:181.
Chu et al., Ann Surg 1999;229:536.
Chu et al., Ann Surg Oncol 1999;6:756.
Cody, Breast Cancer Res 2001;3:104.
Cox, J. Royal Statistical Society 1972;B34:187.
Cristianni et al., An Introduction to Support Vector Machines, Cambridge University Press 2000.
Czemiecki et al., Cancer 1999;85:1098.
Dowlatshahi et al., Cancer 1997;80:1188.
Dreiseitel et al., J Biomed Inform 2002;35:352.
Giuliano et al., J Clin Oncol 2000;18:2553.
Giuliano et al.,. Clin Oncol 1997; 15:2345.
Harrell et al., In: Regressions Modeling Strategies With Applications to Linear Models, Logistic Regression, and Survival Analysis. New York: Springer-Verlag; 2001.
Harrell et al., Stat Med 1996; 15:361.
Hastie, The Elements of Statistical Learning, Springer (2001).
Hauben et al., Drug Saf 2003:26:159.
Hayward et al., Arch Surg 1987;122:1244.
Jakub et al., Am J Surg 2002;184: 302.
Kamath et al., Arch Surg 2001;136:688.
Krag et al., N Engl J Med 1998;339:941.
Moore et al., J Surg Oncol 1997;66:2.
O'Hea et al., J Am Coll Surg 1998;186:423.
Orr, Ann Surg Oncol 1999;6:109.
Osteen et al., Breast Cancer Res Treat 1985;5:17.
Province et al., Adv Genetics 2001;42:273.
Rahusen et al., Arch Surg 2001; 136:1059.
Reynolds et al., J Clin One 1999;17:1720.
Ross et al., J. Virol. 2001; 165:1562.
Ross et al., Semin Urol Oncol 2002;20:82.
Rumelhart et al., (eds), Parallel Distributed Processing: Exploration in the Microstructure of Cognition Volume 1, Foundations Cambridge, Mass., The MIT Press (1986).
Sachdev et al., Am J Surg 2002;183:213.
SAS/STAT User Manual Version 9. 2003; The SAS Institute, Cary, N.C.
Sosa et al., Ann Surg Oncol 1998;5:140.
Tan et al., Proc Am Soc Clin Onc 2002. Abstract 146.
Temple et al, Ann Surg Oncol 2002; 9:654.
Teng et al., Am Surg 2000;66:574.
Turner et al., Ann Surg 1997;226:271.
Turner et al., Cancer 2000;89:574.
Veronesi et al., J Natl Cancer Inst 1999;91:368.
Veronesi et al., Lancet 1997;349:1864.
Viale et al., Cancer 2001;92:1378.
Weiser et al., Ann Surg Oncol 2001;8:145.
Wong et al., Arch Surg 2001;136:563.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to determine a likelihood of additional nodal metastases in a breast cancer patient with a positive sentinel node biopsy, comprising:
a) detecting or determining two or more factors of the patient including pathological size of the breast carcinoma, number of positive sentinel lymph nodes, number of negative sentinel lymph nodes, method for histopathologic evaluation of the sentinel lymph node, tumor type and nuclear grade, estrogen receptor status, presence or absence of lymphovascular invasion, and/or presence or absence of multifocality in the primary tumor; and
b) correlating two or more of the factors to the likelihood of additional nodal metastases in the patient.

2. A method to determine a likelihood of additional nodal metastases in a breast cancer patient with a positive sentinel node biopsy, comprising:
a) inputting test information to a data input means, wherein the information comprises two or more factors of the patient including pathological size of the breast carcinoma, number of positive sentinel lymph nodes, number of negative sentinel lymph nodes, method of histopathologic evaluation of the sentinel lymph node, tumor type and nuclear grade, estrogen receptor status, presence or absence of lymphovascular invasion, and/or presence or absence of multifocality;
b) executing a software for analysis of the test information; and
c) analyzing the test information so as to provide the likelihood of additional nodal metastases in the patient.

3. A method for predicting a likelihood of additional nodal metastases in a breast cancer patient with a positive sentinel node biopsy, comprising:
a) correlating two or more factors for the patient to a functional representation of two or more factors determined for each of a plurality of persons previously diagnosed with invasive breast cancer, having a positive sentinel lymph node biopsy and subjected to completion axillary lymph node dissection, so as to yield a value for total points for the patient, which factors for each of the plurality of persons is correlated with the likelihood of additional nodal metastases for each person in the plurality, wherein the two or more factors include pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or presence or absence of multifocality, wherein the functional representation comprises a scale for two or more of the factors, a total points scale, and a predictor scale, wherein the scales for the pathological size of the carcinoma, number of positive sentinel lymph nodes, number of negative sentinel lymph nodes, method for histopathogic evaluation of the sentinel lymph nodes, tumor type and nuclear grade, estrogen receptor status, lymphovascular invasion, and multifocality, each have values on the scales which can be correlated with values on the points scale, and wherein the total points scale has values which may be correlated with values on the predictor scale; and
b) correlating the value on the total points scale for the patient with a value on the predictor scale to predict the likelihood of additional nodal metastases in the patient.

4. The method of claim 3 wherein the functional representation is a nomogram.

5. The method of claim 1 which includes detecting or determining the pathological size of the carcinoma, number of positive sentinel lymph nodes, number of negative sentinel lymph nodes, and method for histopathogic evaluation of the sentinel lymph nodes.

6. The method of claim 2 wherein the information is the pathological size of the carcinoma, number of positive sentinel lymph nodes, number of negative sentinel lymph nodes, and method for histopathogic evaluation of the sentinel lymph nodes.

7. The method of claim 3 wherein the factors are the pathological size of the carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion and present or absence of multifocality.

8. The method of claim 1 or 3 wherein the correlating is conducted by a computer.

9. An apparatus, comprising:
a data input means, for input of test information for two or more factors from a breast cancer patient with a positive sentinel node biopsy, which factors include pathological size of the carcinoma, number of positive sentinel lymph nodes, number of negative sentinel lymph nodes, method for histopathogic evaluation of the sentinel lymph nodes, tumor type and nuclear grade, estrogen receptor status, the presence or absence of lymphovascular invasion, and/or the presence or absence of multifocality in the primary tumor;
a processor, executing a software for analysis of each factor;
wherein the software analyzes the factors for the patient and provides a likelihood of additional nodal metastases in the patient.

10. The apparatus of claim 9 wherein the test information is input manually using the data input means.

11. The apparatus of claim 9 wherein the software constructs a database of the test information.

12. The apparatus of claim 9 wherein the factors are the pathological size of the carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion and present or absence of multifocality.

13. An apparatus for predicting a likelihood of additional nodal metastases in a breast cancer patient with a positive sentinel node biopsy, which apparatus comprises:
a) a correlation of two or more factors for each of a plurality of persons previously diagnosed with invasive breast cancer, having a positive sentinel lymph node biopsy and having completion axillary lymph node dissection, with a likelihood of additional nodal metastases for each person of the plurality of persons, wherein the two or more factors include pathological size of the invasive carcinoma, number of positive sentinel lymph nodes, number of negative sentinel lymph nodes, method for histopathogic evaluation of the sentinel lymph nodes, tumor type and nuclear grade, estrogen receptor status, the presence or absence of lymphovascular invasion, and/or the presence or absence of multifocality of the primary tumor; and
b) a means for comparing an identical set of factors determined from a patient having breast cancer and a positive sentinel lymph node biopsy to the correlation to predict a likelihood of additional nodal metastases in the patient.

14. The apparatus of claim 13 wherein the factors are the pathological size of the carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion and present or absence of multifocality.

15. A nomogram for the graphic representation of a likelihood of additional nodal metastases in a breast cancer patient with a positive sentinel node biopsy, comprising: a plurality of scales and a solid support, the plurality of scales being disposed on the support and comprising a scale for two or more of pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or multifocality of the primary tumor, a points scale, a total points scale and a predictor scale, wherein the two or more scales for two or more of pathological size of the breast carcinoma, number of positive sentinel lymph nodes, number of negative sentinel lymph nodes, method for histopathogic evaluation of the sentinel lymph nodes, the presence or absence of lymphovascular invasion, and/or the presence or absence of multifocality, each has values on the scales, and wherein the scales for the pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or the presence or absence of multifocality, are disposed on the solid support with respect to the points scale so that each of the values on the pathological size, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or the presence or absence of multifocality, can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, or the presence or absence of multifocality of the patient, can be added together to yield a total points value, and the total points value can be correlated with the predictor scale to predict the likelihood of additional nodal metastases in the breast cancer patient with a positive sentinel node biopsy.

16. The nomogram of claim 15 wherein the solid support is a laminated card.

17. A method to predict a likelihood of additional nodal metastases in a breast cancer patient with a positive sentinel lymph node biopsy comprising:
determining two or more factors for a patient, which two or more factors include pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or presence or absence of multifocality of the primary tumor, matching the factors to the values on the scales of the nomogram of claim 11; determining a separate point value for each of the factors; adding the separate point values together to yield a total points value; and correlating the total points value with a value on the predictor scale of the nomogram to determine the likelihood of additional nodal metastases in the patient.

18. An apparatus for predicting a likelihood of additional nodal metastases in a breast cancer patient with a positive sentinel node biopsy, which apparatus comprises: a scale for two or more factors of the patient which include pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or the presence or absence of multifocality in the primary tumor, a points scale, a total points scale and a predictor scale, wherein the scales for pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or presence or absence of multifocality, each has values on the scales, wherein the scales for pathological size of the breast carcinoma, number of positive sentinel lymph nodes, number of negative sentinel lymph nodes, method for histopathogic evaluation of the sentinel lymph nodes, tumor type and nuclear grade, estrogen receptor status, lymphovascular invasion, and/or multifocality, are disposed so that each of the values for the pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or presence or absence of multifocality, can be correlated with values on the points scale, wherein the total points scale has values on the total points scale, and wherein the total points scale is disposed on the solid support with respect to the predictor scale so that the values on the total points scale may be correlated with values on the predictor scale, such that the values on the points scale correlating with the pathological size of the breast carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or presence or absence of multifocality, can be added together to yield a total points value, and the total points value can be correlated with the predictor scale to predict the likelihood of additional nodal metastases in the patient.

19. A system comprising:
a processor;
an input device;
an output device;
a storage device;
a database wherein the database includes data collected from a plurality of patients previously diagnosed with invasive breast cancer, having a positive sentinel lymph node biopsy and subjected to completion axillary lymph node dissection; software operable on the processor to:
receive input from the input device, the input including two or more factors for determining a likelihood of additional nodal metastases in a breast cancer patient with a positive sentinel node biopsy, wherein the two or more factors include pathological size of the breast carcinoma, the number of positive sentinel lymph nodes, the number of negative sentinel lymph nodes, method for histopathologic evaluation of the sentinel lymph node, tumor type and nuclear grade, estrogen receptor status, presence or absence of lymphovascular invasion, and/or presence or absence of multifocality in the primary tumor; and
correlate received input with the collected data from the plurality of patients to determine the likelihood of additional nodal metastases the patient.

20. The system of claim 19 further comprising a network connection.

21. The system of claim 20 wherein the network is the internet.

22. The system of claim 19 where the database is a relational database management system.

23. The system of claim 19 wherein the output device is a video display.

24. The system of claim 19 wherein the output device is a printer.

25. The system of claim 19 wherein the system is a personal computer.

26. The system of claim 19 wherein the system is a handheld computing device.

27. The system of claim 26 wherein the handheld computing device includes PalmOS.

28. The system of claim 20 wherein the database is accessible via the network.

29. The system of claim 21 wherein the system accepts input and provides output over the internet.

30. The system of claim 29 wherein the input is received and the output is provided in a markup language.

31. The system of claim 30 wherein the markup language is HTML.

32. The system of claim 19 wherein the factors are the pathological size of the carcinoma, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion and present or absence of multifocality.

33. A machine-readable medium having instructions thereon for causing a suitably configured information-handling system to perform the method of claim 1, 2, 3, 4, 5, or 6.

34. A system for predicting a likelihood of additional nodal metastases in a breast cancer patient with a positive sentinel lymph node biopsy, the system comprising:
a data structure for storing historic breast cancer data, the structure contained in a memory and comprising a plurality of factors each corresponding to a characteristic of breast cancer; and
a processing device including program means for correlating two or more factors corresponding to characteristics of breast cancer with factor data collected from a patient with a positive sentinel lymph node biopsy, wherein the correlating results in a likelihood of additional nodal metastases in the patient which is output by the processing device.

35. The system of claim 34 wherein the two or more factors include pathological size of the invasive tumor, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, and/or presence or absence of multifocality in the primary tumor.

36. A method for operating an information-processing device comprising:
maintaining a database of historic data wherein the historic data includes a plurality of scored factors corresponding to a plurality of patients diagnosed with breast cancer, having a positive sentinel lymph node biopsy, and subjected to completion axillary lymph node dissection;
collecting scores from a current patient with a positive sentinel lymph node biopsy for the plurality of factors; and
correlating the scores collected from the current patient with the historic data to determine the likelihood of additional nodal metastases in the patient.

37. The method of claim 36 wherein the two or more factors include pathological size of the invasive tumor, number of positive sentinel lymph nodes, number of negative sentinel lymph nodes, method for histopathogic evaluation of the sentinel lymph nodes, tumor type and nuclear grade, estrogen receptor status, presence or absence of lymphovascular invasion, or presence or absence of multifocality in the primary tumor.

38. The method of claim 1, 2, 3, 17 or 37 wherein the method of detection is immunohistochemistry.
